(19) United States
(12) Patent Application Publication
(10) Pub. No.: US 2009/0209944 A1
(43) Pub. Date: Aug. 20, 2009

(54) COMPONENT OF AN IMPLANTABLE MEDICAL DEVICE COMPRISING AN OXIDE DISPERSION STRENGTHENED (ODS) METAL ALLOY

(75) Inventor: James M. Carlson, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/369,552

(22) Filed: Feb. 11, 2009

Related U.S. Application Data
(60) Provisional application No. 61/028,702, filed on Feb. 14, 2008.

(51) Int. Cl. A61M 25/098; C22C 49/00; A61M 5/00; A61B 17/22; A61F 2/82

(52) U.S. Cl. 604/529; 420/591; 604/264; 606/200; 623/1.34

(57) ABSTRACT

An implantable medical device includes, according to one embodiment, at least one radiopaque and MRI-compatible component comprising an oxide dispersion strengthened metal alloy, where the oxide dispersion strengthened metal alloy has a volume magnetic susceptibility of no greater than about 100×10⁻⁶ and a radiopacity greater than that of grade 304 stainless steel.

### RELATED APPLICATION

The present patent document claims the benefit of the filing date under 35 U.S.C. §119(e) of U.S. Provisional Patent Application Ser. No. 61/028,702, which was filed on Feb. 14, 2008, and is hereby incorporated by reference in its entirety.

### TECHNICAL FIELD

The present disclosure relates generally to medical devices and more particularly to implantable medical devices comprising radiopaque and MRI-compatible components.

### BACKGROUND

Molybdenum (Mo) is a refractory metal known for its high melting temperature (Tₘ˜2625° C.); it has the sixth highest melting temperature of any of the elements. Accordingly, molybdenum and its alloys are widely used in high-temperature applications, such as in nuclear fuel processing, missile and aircraft parts, glass furnace electrodes, nuclear power and propulsion systems, flame-resistant coatings, and others.

Oxide-dispersion strengthened (ODS) molybdenum alloys are molybdenum-based materials including a pure or alloyed molybdenum matrix with very fine oxide particles dispersed therein. The presence of the oxide particles is believed to enhance the thermal stability of the molybdenum alloy at elevated temperatures. For example, U.S. Pat. Nos. 5,868,876 and 6,102,979, which are hereby incorporated by reference in their entirety, disclose high-strength, high-temperature creep-resistant ODS molybdenum alloys designed for stable use at operating temperatures above 0.55 Tₘ.

### BRIEF SUMMARY

An implantable medical device including at least one radiopaque and MRI-compatible component is described herein. The component includes, according to one embodiment, an oxide dispersion strengthened metal alloy having a volume magnetic susceptibility of no greater than about 100×10⁻⁶ and a radiopacity greater than that of grade 304 stainless steel. According to another embodiment, the radiopaque and MRI-compatible component comprises an oxide dispersion strengthened metal alloy including oxide particles at a concentration of about 1 vol. % or less.

### BRIEF DESCRIPTION OF THE DRAWINGS

FIG. 1A is a radiograph of a wire made of grade 304V stainless steel;

FIG. 1B is a radiograph of a wire made of Mo-0.8 vol. % La₂O₃; and

FIG. 2 is a schematic of a component of a medical device including an oxide dispersion strengthened metal alloy.

### DETAILED DESCRIPTION

### Definitions

The term “implantable medical device” refers to a medical device that is either permanently or temporarily inserted into a patient's body for treatment of a medical condition.

The term “oxide dispersion strengthened metal alloy,” or “ODS metal alloy,” refers to a metal alloy that has a substantially pure or alloyed metal matrix with oxide particles dispersed therein.

The term “magnetic susceptibility” refers to a ratio of the magnetization of a material in response to an applied magnetic field.

The term “radiopacity” refers to the capacity to absorb x-ray radiation.

An implantable medical device including at least one component comprising an oxide dispersion strengthened (ODS) metal alloy is described herein. The component comprising the ODS metal alloy can be viewed in vivo using x-ray fluoroscopy and is sufficiently compatible with magnetic resonance imaging (MRI) to allow substantially artifact-free MRI scans and images to be obtained.

X-ray fluoroscopy and magnetic resonance imaging (MRI) are well known and useful techniques for imaging structures within the body. X-ray fluoroscopy is commonly employed to track the position of an implantable medical device during intraluminal delivery to a treatment site. MRI can be used as a diagnostic tool to visualize tissue, blood flow and other structures in the vicinity of the implanted device. The success of each of these techniques in providing the desired images is at least partly dependent on the characteristics of the material(s) that compose the implanted device.

Some materials, such as stainless steel, do not provide sufficient x-ray absorption (radiopacity) for unambiguous device visualization during x-ray fluoroscopy. Also, stainless steel and other ferromagnetic or strongly paramagnetic materials may introduce artifacts into MRI scans that hinder visualization and diagnostics.

The inventor has discovered that ODS metal alloys, in particular, ODS molybdenum alloys, have a surprisingly suitable combination of magnetic, x-ray absorption, and mechanical properties for use in medical device applications at body temperature. In particular, the MRI compatibility, radiopacity, formability and tensile strength of ODS molybdenum alloys comprising a molybdenum matrix and 0.8 vol. % La₂O₃ particles, as will be discussed further below, suggest that such alloys may be advantageously employed as medical device materials.

The ODS metal alloy of the radiopaque and MRI-compatible component described herein includes a pure or alloyed metal matrix and oxide particles dispersed therein. The metal matrix of the ODS metal alloy may include molybdenum, rhenium, tungsten, hafnium, gold, platinum, palladium, silver, or another metallic element. The metallic element is preferably a refractory metal or a precious metal.

The matrix of the ODS metal alloy may include molybdenum, for example. According to one embodiment, the ODS metal alloy includes a substantially pure molybdenum matrix that contains unalloyed molybdenum and any incidental impurities. According to another embodiment, the matrix of the ODS metal alloy includes molybdenum and at least one other metallic alloying element, such as, for example rhenium, at a concentration of about 49 wt. % or less. For example, the matrix may be a molybdenum-rhenium alloy including from about 1 wt. % Re to about 48 wt. % Re. In another example, the matrix may be a molybdenum-rhenium alloy including from about 5 wt. % Re to about 25 wt. % Re.

The oxide particles (i.e., dispersions or dispersoids) present in the ODS metal alloy may include any metal oxide or semiconducting oxide. Preferably, the particles include a rare earth metal oxide, such as lanthanum oxide, cerium oxide, yttrium oxide or thorium oxide. The oxide particles are preferably present at a concentration of about 5 vol. % or less. The oxide particles may also be present at a concentration of about 1 vol. % or less. For example, the oxide particles may be present at a concentration of about 0.8 vol. %. Suitable ODS alloys including, for example, 0.7 wt. % La₂O₃ or 0.3 wt. % La₂O₃ in a molybdenum matrix, are commercially available from Plansee SE of Reutte, Austria and other manufacturers. The oxide particles generally range in size from a few nanometers to a few microns.

Due to the high melting temperature of molybdenum, ODS molybdenum alloys are generally manufactured using powder metallurgy methods (e.g., powder mixing and cold/hot isostatic pressing followed by sintering) and thermomechanical processing.

### MRI Compatibility

MRI compatible materials generally have a low magnetic susceptibility. The magnetic susceptibility Xₘ of a material can be represented by the ratio of the magnetization M of the material to the applied magnetic field H:

$${X}_{m} = \frac{M}{H}$$

Different materials respond differently to applied magnetic fields H, and thus may have widely varying values of magnetic susceptibility Xₘ. Materials are classified as diamagnetic, paramagnetic, or ferromagnetic depending on their response to an applied magnetic field.

A diamagnetic material has a small and negative magnetic susceptibility. In response to an applied field, a diamagnetic material has a weak net magnetic moment in a direction perpendicular to the applied field. Outside of the applied magnetic field, the material has no net magnetic moment. Paramagnetic materials exhibit a small and positive magnetic susceptibility. Such materials have randomly oriented magnetic moments that slightly align in response to a magnetic field to achieve a low magnetization in the same direction as the field. Paramagnetic materials do not remain magnetized when the external field is removed. Ferromagnetic materials have a large and positive magnetic susceptibility. The atoms of ferromagnetic materials have parallel, aligned magnetic moments that exist even in the absence of an applied magnetic field.

As Equation 1 indicates, materials that respond strongly to a magnetic field have a high magnetic susceptibility. In the case of medical devices having components made of such materials (e.g., stainless steel, which is ferromagnetic), a disturbance in the magnetic field is created around the component when the component is visualized with MRI. As a result, the component may not properly transfer MRI signal information and portions of the magnetic resonance image or scan in the vicinity of the component may become distorted. The distortion can result in severe dark or black areas in the image. Accordingly, materials having a high magnetic susceptibility are generally not believed to be MRI-compatible.

Magnetic measurements to determine the mass and volume magnetic susceptibility (X_{M} and X_{V}) of an ODS molybdenum alloy were carried out using a commercially available magnetic susceptibility balance (MSB-MK1, Alpha Aesar/Johnson Matthey, West Chester, Pa.). The MSB-MK1 balance utilizes a stationary sample and moving magnets. Two pairs of magnets are placed at opposite ends of a beam to make a balanced system. Introduction of the sample causes a deflection of the beam, and the movement is optically detected. A compensating force is applied by a coil between the other pair of magnets. The current required to maintain equilibrium of the balance beam is proportional to the force exerted by the sample. This method, which is referred to as the Evans method, is an updated version of the traditional technique (Gouy method) of measuring magnetic susceptibility, which employs a conventional laboratory balance and large permanent magnets that remain stationary while the sample is caused to move.

The mass and volume susceptibilities X_{M} and X_{V} can be determined by using the MSB-MK1 magnetic susceptibility balance in conjunction with the following equations:

X_{M}=C·L·(R−Rₒ)/(10⁹·m), where

C=calibration constant (1.115 for the present apparatus)

L=length of sample in centimeters

m=mass of sample in grams

R=balance reading for sample in tube

Rₒ=balance reading for empty tube; and

X_{V}=X_{M}·ρ, where ρ is the density of the material.

The above procedure was applied to a Mo-0.8 vol. % La₂O₃ wire specimen to determine values of X_{M} and X_{V}, which are presented in Table 1 below. For comparison, measured values of X_{M} and X_{V} for 304V stainless steel wire and a superelastic nickel-titanium alloy wire, both of which are presently used in medical devices, are also provided in the table. Several measurements were taken for each specimen, and the average of the measured values is presented in the table. All of the wire specimens were approximately 1 inch (2.54 cm) long. To the inventor's knowledge, the data provided in Table 1 for the ODS molybdenum alloy are not available elsewhere in the literature or otherwise known.

The data indicate that Mo-0.8 vol. % La₂O₃wire has a mass magnetic susceptibility X_{M} of 8.95×10⁻⁶ cm³/g, which is about 2.5% of that of grade 304V stainless steel wire, and a volume magnetic susceptibility X_{V} of 91.5×10⁻⁶, which is about 2.9% of that of grade 304V stainless steel.

TABLE 1
Magnetic Susceptibility Data

| | X_{M} | X_{V} |
|---|---|---|
| Alloy | (×10⁻⁶ cm³/g) | (×10⁻⁶) |
| Mo—0.8% La₂O₃ | 8.95 | 91.5 |
| Superelastic Ni—Ti | 12.48 | 81.1 |
| Grade 304V stainless steel | 360.0 | 3,132 |

For MRI-compatibility, the medical device of the present disclosure preferably includes a component made of an ODS metal alloy having a volume magnetic susceptibility in the range of from about −50×10⁻⁶ to about 200×10⁻⁶. More preferably, the volume magnetic susceptibility is in the range of from about −50×10⁻⁶ to about 100×10⁻⁶. A medical device including such a component may be visualized using MRI procedures without creating an imaging artifact, thus allowing the device and surrounding tissue to be properly and accurately imaged.

### Radiopacity

The intensity of x-rays transmitted through a material Iₓ is related to the incident intensity I₀, material thickness x, and the linear absorption coefficient μ:

Iₓ=I₀e^{-μx}

The radiopacity of a material is related to its linear absorption coefficient, μ, which depends on the effective atomic number (Z_{eff}) and density (ρ) of the material, and on the energy (E) of the incoming x-ray photons:

$$\frac{μ}{ρ} = const \cdot \frac{{Z}_{eff}^{3}}{{E}^{3}}$$

The linear absorption coefficient μ is proportional to the density ρ of the material, and thus the quantity μ/ρ is a material constant known as the mass absorption coefficient and expressed in units of cm²g⁻¹.

Materials or tissues that substantially transmit incident x-rays are not readily visible in x-ray images. In contrast, radiopaque materials absorb incident x-rays over a given energy range and tend to show high contrast and good visibility in x-ray images. The magnitude of the linear absorption coefficient of a material may be a good indicator of its capacity for absorbing x-ray radiation, and thus its x-ray opacity or radiopacity.

Experimentally, relative values of radiopacity may be obtained by comparing the x-ray contrast of different specimens. X-ray contrast can be calculated by collecting x-ray intensity data from a specimen using an x-ray fluoroscope or similar radiological apparatus and subtracting the transmitted intensity from the background intensity at a given voltage.

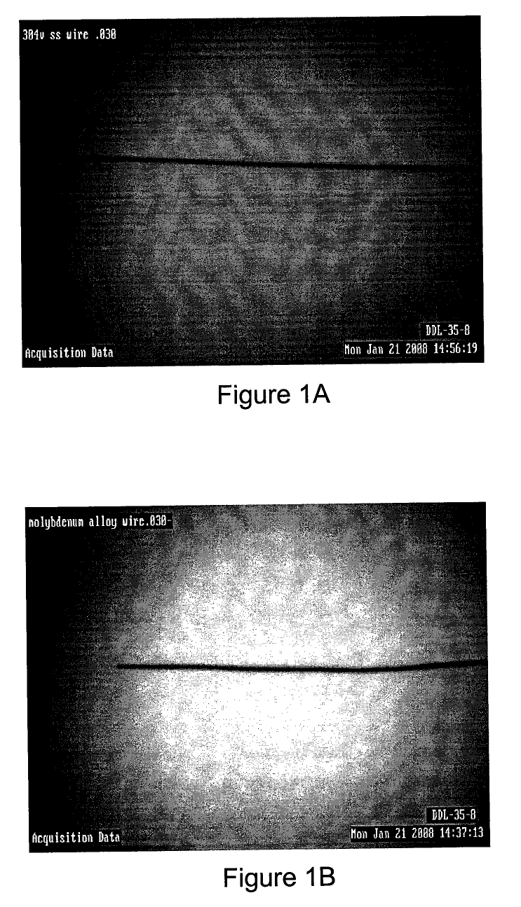

FIGS. 1A and 1B illustrate the relative contrast of x-ray images of wires of 0.030 (˜0.076 cm) in diameter made of Mo-0.8 vol. % La₂O₃ and grade 304V stainless steel at a voltage of 55 keV. FIG. 1A is an radiograph of the stainless steel wire and FIG. 1B is a radiograph of the ODS molybdenum wire. Shown in Table 2 are approximate values of x-ray contrast determined for the wire specimens based on the grayscale ratings for the radiographs. The density of each material is also indicated.

The x-ray contrast values indicate that the ODS molybdenum alloy (Mo-0.8 vol. % La₂O₃) has a radiopacity that exceeds that of grade 304V stainless steel by at least about 35%. The ODS molybdenum alloy is also about 50% more radiopaque than superelastic nickel-titanium (Nitinol).

TABLE 2
X-Ray Contrast Values

| Alloy | Density (g/cm³) | X-ray contrast |
|---|---|---|
| Mo—0.8 vol. % La₂O₃ | 10.2 | 1.35 |
| 304V stainless steel | 8.7 | 1.00 |

Preferably, the medical device of the present disclosure includes a component made of an ODS metal alloy having a radiopacity higher than that of grade 304 stainless steel by at least about 15%. More preferably, radiopacity of the component is at least about 25% higher than that of grade 304 stainless steel. A medical device including such a component may be accurately and clearly visualized during delivery and/or manipulation in a body vessel using x-ray fluoroscopy.

### Formability and Mechanical Behavior

The ODS metal alloy of the present disclosure preferably has adequate formability to be worked into the desired shape of the medical device component. In addition, the component comprising the ODS metal alloy preferably has sufficient strength and ductility to perform the desired functions in the body.

The formability and mechanical properties of a Mo-0.8 vol. % La₂O₃ alloy specimen were evaluated through a series of wire drawing experiments and tensile tests. Wire drawing is a forming (or working) operation that causes a reduction in the diameter of a wire specimen. Due to the effects of strain hardening, wire drawing also generally produces an increase in the strength of the wire specimen and a concomitant decrease in ductility.

Annealed Mo-0.8 vol. % La₂O₃ wire of 0.0299 inch (0.0760 cm) in diameter was subjected to a series of drawing operations to reduce the diameter by over 70% to 0.0079 inch (0.0201 cm). To obtain this reduction, the Mo-0.8 vol. % La₂O₃ wire made 12 passes through a die. The change in diameter of the wire with each pass is recorded in Table 3 below.

Surprisingly, the Mo-0.8 vol. % La₂O₃ wire was found to be sufficiently ductile to undergo 12 consecutive drawing passes with no interpass anneals (heat treatments). Interpass anneals are commonly employed to reduce strain hardening effects during wire drawing, thereby preventing fracture and allowing for continued drawing. The inventor believes such formability may be particularly advantageous for production-scale drawing operations since interpass annealing steps can be expensive and time-consuming.

TABLE 3
Wire Drawing Data

| | Diameter | Diameter | Reduction |
|---|---|---|---|
| Pass # | in | cm | in Diameter % |
| 0 | 0.0299 | 0.0760 | — |
| 1 | 0.0285 | 0.0724 | −4.7 |
| 2 | 0.0253 | 0.0643 | −15.4 |
| 3 | 0.0226 | 0.0575 | −24.3 |
| 4 | 0.0201 | 0.0510 | −32.9 |
| 5 | 0.0179 | 0.0454 | −40.3 |
| 6 | 0.0159 | 0.0403 | −47.0 |
| 7 | 0.0143 | 0.0364 | −52.1 |
| 8 | 0.0127 | 0.0321 | −57.8 |
| 9 | 0.0114 | 0.0288 | −62.1 |
| 10 | 0.0100 | 0.0255 | −66.4 |
| 11 | 0.0090 | 0.0228 | −70.0 |
| 12 | 0.0079 | 0.0201 | −73.6 |

Tensile tests of the Mo-0.8 vol. % La₂O₃ wire were carried out at room temperature in accordance with American Society of Testing and Materials (ASTM) E8-04 “Standard Test Methods for Tension Testing of Metallic Materials,” which is hereby incorporated by reference in its entirety. A tensile test was conducted on a sample of the annealed wire prior to drawing and then on a sample of the wire after each drawing pass. Consequently, a total of 13 tensile tests were carried out. The yield strength, ultimate tensile strength, percent elongation and ratio of the yield strength to the tensile strength (Y/T) are presented in Table 4 below for each sample tested.

As is generally known to those of skill in the art, the tensile strength (i.e., ultimate tensile strength or UTS) of a material corresponds to the maximum engineering stress that can be sustained by the material in tension without fracture. Engineering stress is defined as

$$\frac{F}{{A}_{0}} ,$$

where F represents tensile force and A₀ represents the original cross-sectional area of the specimen prior to application of the force. The yield strength corresponds to the stress required to produce a small amount of plastic strain (permanent deformation) in the material, which can be seen as a departure from linearity on a stress-strain curve. Typically, a straight line is constructed parallel to the linear portion of the stress-strain curve at a strain offset of 0.002 (0.2%) to determine the point of yielding, and thus the yield strength.

TABLE 4
Mechanical Properties of Mo - 0.8 vol. % La₂O₃ Wire

| | Yield | Yield | Tensile | Tensile | | |
|---|---|---|---|---|---|---|
| | Strength | Strength | Strength | Strength | | |
| Pass # | ksi | MPa | ksi | MPa | Elongation % | Y/T |
| 0 | 136.9 | 943.9 | 142.9 | 985.0 | 19.7 | 0.96 |
| 1 | 144.5 | 996.5 | 151.0 | 1041.1 | 14.6 | 0.96 |
| 2 | 163.8 | 1129.6 | 168.0 | 1158.5 | 10.3 | 0.98 |
| 3 | 172.9 | 1192.2 | 176.9 | 1219.7 | 6.8 | 0.98 |
| 4 | 182.4 | 1257.5 | 187.3 | 1291.7 | 5.3 | 0.97 |
| 5 | 187.3 | 1291.5 | 194.6 | 1341.6 | 5.9 | 0.96 |
| 6 | 196.9 | 1357.7 | 207.1 | 1428.0 | 5.7 | 0.95 |
| 7 | 208.5 | 1437.5 | 217.8 | 1501.9 | 5.4 | 0.96 |
| 8 | 222.8 | 1536.5 | 232.1 | 1600.6 | 5.2 | 0.96 |
| 9 | 234.7 | 1618.6 | 245.2 | 1690.4 | 4.8 | 0.96 |
| 10 | 248.2 | 1711.3 | 263.3 | 1815.3 | 4.4 | 0.94 |
| 11 | 273.7 | 1887.4 | 278.3 | 1918.7 | 4.7 | 0.98 |
| 12 | 277.3 | 1912.2 | 298.5 | 2058.2 | 3.9 | 0.93 |

According to one embodiment, a component comprising the ODS metal alloy has a yield strength of at least about 1430 MPa (˜207 ksi) and a tensile strength of at least about 1500 MPa (˜218 ksi). More preferably, the component has a yield strength of at least about 1900 MPa (˜275 ksi) a tensile strength of at least about 2050 MPa (˜297 ksi). It is also preferred that the elongation to failure is at least about 4%, or at least about 5%. It would be further advantageous for some applications if the elongation to failure is at least about 10%.

### Medical Devices

A medical device in accordance with the present disclosure includes at least one MRI-compatible and radiopaque component comprising an ODS metal alloy. As described above, the ODS metal alloy is preferably an ODS molybdenum alloy. The component may be formed in whole or in part of the ODS metal alloy from wire, tubing, ribbon, button, bar, disk, sheet, foil, or another pressed, cast, or worked shape.

According to one embodiment, the component including the ODS metal alloy described herein is a wire. According to another embodiment, the component is a tube or ring (i.e., “cannula”). The wire or cannula may be formed by extrusion and/or drawing methods known in the art. Gun drilling may be used to form a hole in an extruded or drawn cylinder. The cannula may also be produced by forming a sheet into a tubular configuration and welding the edges.

It is contemplated that the component may have a composite structure in which one or more portions of the structure are formed of the ODS metal alloy, and one or more portions of the structure are formed of a different material. For example, the component may include distinct constituents, such as layers, cladding, filaments, strands, cables, particles, fibers, and/or phases, where one or more of the constituents are formed from the ODS metal alloy, and one or more are formed from the different material. Such a composite structure may provide a component having improved MRI compatibility, radiopacity and/or mechanical properties compared to a monolithic component.

According to one exemplary embodiment, the component is a wire having a composite structure. For example, the wire may include a core layer and one or more outer layers disposed about the core layer. One or more of the layers may be formed of the ODS metal alloy, and one or more of the layers may be formed of a different material. The wire can be formed by drawing or extruding a preform including multiple coaxial layers to produce the composite structure. Alternatively, the wire may be formed by coating one or more layers on a core layer by plating or another deposition technique.

According to another exemplary embodiment, the component is a cannula having a composite structure. For example, the cannula may be formed from a multilayered tube including one or more coaxial layers of the ODS metal alloy and one or more coaxial layers of another material. The multilayered tube may be formed by drawing or extruding coaxial tubing. Alternatively, the multilayered tube may be prepared from a clad sheet that has been formed into a tube.

The component may be employed individually or in combination with other components to form an implantable medical device, such as, for example, a stent, a stent graft, a wire guide, a radiopaque marker or marker band, a torqueable catheter, an introducer sheath, an orthodontic arch wire, or a manipulation, retrieval, or occlusive device such as a grasper, a snare, a basket (e.g., stone extraction or manipulation basket), a vascular plug, an embolization coil, or an embolic protection filter.

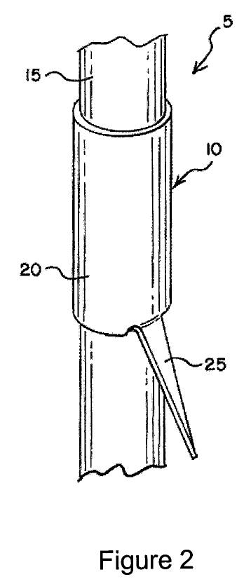

According to one embodiment, the medical device is a stent. The stent may be self-expanding or balloon-expandable. All or a portion of the stent may be made of the ODS metal alloy. The stent may further include a graft material attached thereto. For example, the device may be a stent graft where components of the device, such as one or more of the stent rings (e.g., the top stent ring), are formed in part or in whole of the ODS metal alloy. An exemplary stent ring 10 comprising the ODS metal alloy is shown in FIG. 2. The stent ring 10 comprises a cannula 20 overlying a portion of a stent 15 and a barb 25 extending from the cannula 20 for penetration into adjacent tissue.

An implantable medical device including at least one MRI-compatible component comprising an oxide dispersion strengthened (ODS) metal alloy has been described. Preferably, the ODS metal alloy is an ODS molybdenum alloy comprising a pure or alloyed molybdenum matrix with oxide particles dispersed therein. The oxide particles have a concentration of about 1 vol. % or less, according to one embodiment, and may be lanthanum oxide (La₂O₃). Preferably, the ODS molybdenum alloy has a volume magnetic susceptibility of no greater than about 100×10⁻⁶. It is also preferred that the ODS molybdenum alloy has a radiopacity greater than that of grade 304 stainless steel by at least about 25%. Accordingly, during delivery and/or manipulation of the medical device in a body vessel, the component may be accurately visualized using x-ray fluoroscopy. In addition, during a diagnostic procedure using magnetic resonance imaging (MRI), the component, surrounding tissue, and/or blood flow may be unambiguously imaged.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. An implantable medical device including at least one radiopaque and MRI-compatible component comprising an oxide dispersion strengthened metal alloy, wherein the oxide dispersion strengthened metal alloy has a volume magnetic susceptibility of no greater than about 100×10⁻⁶ and a radiopacity greater than that of grade 304 stainless steel.

2. The implantable medical device of claim 1, wherein the radiopacity of the oxide dispersion strengthened metal alloy exceeds that of grade 304 stainless steel by about 25%.

3. The implantable medical device of claim 1, wherein the oxide dispersion strengthened metal alloy comprises a matrix including molybdenum.

4. The implantable medical device of claim 3, wherein the matrix further includes rhenium.

5. The implantable medical device of claim 1, wherein the oxide dispersion strengthened metal alloy includes oxide particles selected from the group consisting of lanthanum oxide, yttrium oxide, cerium oxide and thorium oxide.

6. The implantable medical device of claim 5, wherein the oxide particles comprise lanthanum oxide.

7. The implantable medical device of claim 1, comprising oxide particles at a concentration of about 1 vol. % or less.

8. The implantable medical device of claim 1, wherein a preform of the component is sufficiently formable to undergo a reduction in linear dimension of at least about 50% without an interpass anneal.

9. The implantable medical device of claim 1, wherein the component has a tensile strength of at least about 1500 MPa.

10. The implantable medical device of claim 1, wherein the component comprises one of a wire and a cannula.

11. The implantable medical device of claim 1 selected from the group consisting of a stent, a stent graft, a wire guide, a filter, an embolization coil, a basket, and a snare.

12. The implantable medical device of claim 11, wherein the component is a top stent ring of the stent graft.

13. The implantable medical device of claim 1 including any two or more of:
the radiopacity of the oxide dispersion strengthened metal alloy exceeding that of grade 304 stainless steel by about 25%;
a matrix of the oxide dispersion strengthened metal alloy including molybdenum;
a matrix of the oxide dispersion strengthened metal alloy including molybdenum and rhenium;
the oxide particles being dispersed in the oxide dispersion strengthened alloy at a concentration of about 1 vol. % or less;
the oxide particles being selected from the group consisting of lanthanum oxide, yttrium oxide, cerium oxide and thorium oxide;
a preform of the component being sufficiently formable to undergo a reduction in linear dimension of at least about 50% without an interpass anneal,
the component having a tensile strength of at least about 1500 MPa;
the component comprising one of a wire and a cannula; and
the implantable medical device being selected from the group consisting of a stent, a stent graft, a wire guide, a filter, an embolization coil, a basket, and a snare.

14. An implantable medical device including at least one radiopaque and MRI-compatible component comprising an oxide dispersion strengthened metal alloy including oxide particles at a concentration of about 1 vol. % or less.

15. The implantable medical device of claim 14, wherein the oxide dispersion strengthened metal alloy comprises a matrix including molybdenum.

16. The implantable medical device of claim 14, wherein the concentration of the oxide particles is about 0.8 vol. %.

17. The implantable medical device of claim 14, wherein the oxide particles are selected from the group consisting of lanthanum oxide, yttrium oxide, cerium oxide and thorium oxide.

18. The implantable medical device of claim 14, wherein the component has a volume magnetic susceptibility of no greater than about 100×10⁻⁶ and a radiopacity greater than that of grade 304 stainless steel.

19. The implantable medical device of claim 14, wherein the component has a tensile strength of at least about 1500 MPa.

20. The implantable medical device of claim 14 including any two or more of:
the oxide dispersion strengthened metal alloy comprising a matrix including molybdenum;
the concentration of the oxide particles being about 0.8 vol. %;
the oxide particles being selected from the group consisting of lanthanum oxide, yttrium oxide, cerium oxide and thorium oxide;
the component having a volume magnetic susceptibility of no greater than about 100×10⁻⁶ and a radiopacity greater than that of grade 304 stainless steel; and
the component having a tensile strength of at least about 1500 MPa.
